(19) United States
(12) Patent Application Publication
(10) Pub. No.: US 2022/0175309 A1
(43) Pub. Date: Jun. 9, 2022

(54) METHODS AND APPARATUS FOR SYNCHRONIZING CARDIOVASCULAR AND RESPIRATORY SYSTEMS WITH STRESS AND EXERTION ANALYSIS

(71) Applicant: VARDAS SOLUTIONS LLC, El Dorado Hills, CA (US)

(72) Inventors: Chad Vardas, El Dorado Hills, CA (US); Alex Jones, Carlsbad, CA (US)

(73) Assignee: VARDAS SOLUTIONS LLC, El Dorado Hills, CA (US)

(21) Appl. No.: 17/436,519

(22) PCT Filed: Apr. 16, 2020
(86) PCT No.: PCT/US20/28598

§ 371 Date: Sep. 3, 2021

Related U.S. Application Data
(60) Provisional application No. 62/814,780, filed on Mar. 6, 2019.

(51) Int. Cl. A61B 5/00; A61B 5/0205; A61B 5/16; G16H 40/63; G16H 50/30

(52) U.S. Cl. CPC A61B 5/486; A61B 5/0205; A61B 5/7246; A61B 5/0816; G16H 40/63; G16H 50/30; A61B 5/165

(57) ABSTRACT

Heart Rate Variability Biofeedback is provided outside of the clinic to benefit athletes and individuals suffering from an array illnesses, including stress, anxiety, depression, COPD, asthma, and hypertension. This therapy is provided to the user by analyzing biometrics, calculating and guiding breathing to a Resonance Frequency, analyzing and providing feedback on stress level, tracking time in Resonance and benefits received, and recommending a therapy regimen.

### CROSS-REFERENCE TO RELATED CASES

The present application claims priority to U.S. Provisional Patent Application No. 62/814,780, entitled “METHOD AND APPARATUS FOR BIOMETRIC MEASUREMENT AND PROCESSING,” filed on Mar. 6, 2019, which is hereby incorporated by reference. The present application is related to the following: U.S. patent application Ser. No. 16/284,955, entitled “MEASURING USER RESPIRATION AT EXTREMITIES,” filed on Feb. 25, 2019; U.S. patent application Ser. No. 16/275,153, entitled “MEASURING USER RESPIRATION AT EXTREMITIES,” filed on Feb. 13, 2019; U.S. patent application Ser. No. 16/006,558, entitled “METHODS AND SYSTEMS FOR PROVIDING A BREATHING RATE CALIBRATED TO A RESONANCE BREATHING FREQUENCY,” filed on Jun. 12, 2018, now U.S. Pat. No. 10,398,350, which is a continuation-in-part of U.S. patent application Ser. No. 15/428,115, entitled “STRESS MANAGEMENT USING BIOFEEDBACK,” filed on Feb. 8, 2017, now U.S. Pat. No. 10,517,531, which claims priority to U.S. Provisional Patent Application No. 62/292,450, entitled “WEARABLE APPARATUS WITH BIOFEEDBACK,” filed on Feb. 8, 2016, each of which is hereby incorporated by reference.

### BACKGROUND

It is a common problem for an individual to suffer from one of these illnesses. By some current estimates, 234 million Americans regularly experience psychological symptoms of stress, 64 million Americans are diagnosed with an anxiety disorder, and 54 million Americans are prescribed antidepressants or anti-anxiety drugs.

The benefits of antidepressants and anti-anxiety drugs are undeniable, but the treatment of stress-related disorders with such medications can have a down-side in the form of drug abuse, addiction and adverse effects.

In the United States alone, it is currently estimated that more than 15 million people abuse prescription drugs and that this abuse results in 45% of the drug-related deaths in the U.S.—more than heroin, methamphetamine, and cocaine combined.

Furthermore, even in the absence of drug abuse, a stigma is often associated with the mere taking of medication for stress or anxiety.

For these reasons it would be desirable to have a system and method to reduce stress and anxiety that did not require drugs, such as antidepressants or anti-anxiety drugs.

Heart Rate Variability Biofeedback (HRVB) is backed by 26 years of evidence-based research for helping athletes and reducing the severity of several conditions, including stress, anxiety, depression, COPD, asthma and hypertension. The current implementation of this therapy involves patients visiting a clinic, having sensors attached to their body and being instructed to breathe to a calculated Resonance Frequency (RF) while the clinician guides them to a state of Resonance. Resonance is a physiological state where the vagus nerve and baroreflex are stimulated. RF is a personal breathing rate that most effectively induces Resonance. Clinical HRVB can have strong efficacy, but it's flawed for the following reasons.

Clinical HRVB is inaccessible outside of the clinic, which makes it challenging for individuals to use the therapy when they need it the most. Furthermore, compliance is typically poor due to both the difficulty in scheduling regular appointments and the high cost associated with receiving this therapy in the clinic.

The invention described herein describes an improved implementation of Heart Rate Variability Biofeedback that is available outside of the clinic and designed for effective compliance.

### BRIEF DESCRIPTION OF THE DRAWINGS

The embodiments are illustrated by way of example and not limitation in the figures of the accompanying drawings, in which like references indicate similar elements, and in which:

FIG. 1 is a flow chart of an embodiment of a first method for determining a Resonance Breathing Frequency;

FIG. 2 is a flow chart of an embodiment of a second method for determining a Resonance Breathing Frequency;

FIG. 3 is a description of an embodiment of a method for measuring and providing feedback on Resonance;

FIG. 4 is a description of an embodiment of methods for measuring and providing feedback on the quantity of Heart Rate Variability Biofeedback benefits received;

FIG. 5 is a description of an embodiment of a first method for analyzing user stress level;

FIG. 6 is a description of an embodiment of a method for providing a personalized therapy regimen;

FIG. 7 is a description of an embodiment of a second method for analyzing user stress level and providing feedback;

FIG. 8 is a description of an embodiment of a third method for analyzing user stress level and providing feedback;

FIG. 9 is a simplified, exemplary block diagram of an embodiment of a system for measuring the respiration of a user;

FIG. 10 is an exemplary block diagram of a computing device from the system of FIG. 9;

FIG. 11 includes front, back, top, bottom, and right views of an embodiment of a biometric analysis device implementing embodiments of the systems and methods disclosed herein;

FIG. 12 is a perspective view of the biometric analysis device of FIG. 11;

FIG. 13 is a first exemplary visual indication of calculated values; and

FIG. 14 is a second exemplary visual indication of calculated values.

### DETAILED DESCRIPTION

Hardware

For the measurement of HRV, stress level, and resonance, at least one of the following sensors is used as an input to the calculation of these metrics. A sensor is defined as procurement of at least one biometric through at least one of hardware and software as opposed to solely a physical piece of hardware.

ECG and respiration derived from impedance pneumography require a point on one side of the heart and the other on the other side to create a closed circuit. Points can be anywhere as long as one is on one side of the heart and the other is on the other side. In our embodiment we use the underside of one wrist, and a finger on the opposite hand. In another embodiment, a chest strap with points on either side of the chest is used. A very precise ADC is used to convert small electrical signals from the surface of the skin to integers that calculations can be run on. In an embodiment, an ADS1292r IC from Texas instruments is used, which is an electrocardiogram—analog to digital converter (ADCs) with a built-in programmable gain amplifier (PGA), internal reference and an onboard oscillator. In this embodiment, this sensor, along with a set of algorithms and hardware modifications allows the measurement of real-time respiration as well.

Electrodermal activity, also known as skin conductance, is the property of the human body that causes continuous variation in the electrical characteristics of the skin and is an indication of psychological or physiological arousal. Electrodermal activity is measured by the change in moisture level across a small patch of skin by checking for a change in resistance over that space. This sensor needs two contact points a small distance away from each other. These sensors can be placed on the hands, or really anywhere on the body as long as the distance is in the correct range. In an embodiment, the contacts are on different fingers of the same hand. In another embodiment, the contacts are 1 cm apart on the wrist. A small voltage is applied between the connection points and voltage is measured across the other using a high precision ADC in order to derive the skin resistance in ohms according to ohm's law. In an embodiment, 0.5 volts are applied to one of the leads, and the resulting voltage is measured in the other lead. In an embodiment, the signal quality can be improved by oscillating the signal voltage and calibrating the system while the current is not being applied and adjusting the real data point by that calibrated point.

The temperature of a user's skin is also an indication of psychological or physiological arousal. An acute stress response corresponds to a short term drop in skin temperature. Skin temperature is measured using any method that can obtain a high precision calculation of temperature on a surface. In an embodiment, the difference in voltage between two inductors of different values is used to derive temperature of a user's skin at a point. In another embodiment, the infrared light emitted from a user's skin is measured using a photodiode, which is proportional to temperature of a surface.

A sudden change in blood pressure is also an indicator of psychological or physiological arousal. In an embodiment, an ECG and photoplethysmogram (PPG) sensor are both used to measure Pulse transit Time (PTT). PTT is the distance between the peak of a QRS complex from an ECG signal and the peak of a PPG wave from a PPG sensor. This value is around 100 ms, and a transform is applied to these values to obtain blood pressure measurements. Upper arm ECG can be used for a single point system using this method. PTT has been shown to correlate with blood pressure. In an embodiment, a combination of PPG sensor and a pressure sensor are used to derive blood pressure. The magnitude of a PPG signal varies by pressure of skin pressing against the sensor and by blood pressure. We use a pressure sensor to factor out that variable to determine a blood pressure value. The user must hold their finger to the sensor with the same amount of pressure the entire reading or it is thrown out. This system is passive and wrist mounted, so the pressure sensor performs a transform on the PPG data in order to normalize the wave overtime and get continuous readings without the need for human intervention.

A photoplethysmogram (PPG) sensor can also be used to measure heart rate. This sensor is currently available in many smartwatches in the market.

In an embodiment, the invention described herein utilizes custom hardware with at least one of the sensors described above. In another embodiment, the invention described herein utilizes hardware readily available in the market such as the Samsung Galaxy Watch Active 2 or Apple Watch. In an embodiment, the hardware readily available in the market includes at least one of the sensors described above.

In an embodiment, the invention described herein utilizes a chest patch, a wrist-worn device, a hand-held device, a cell phone, a computer, an automobile, a tablet, a head worn device, electronics worn in the ear, smart glasses, or other implementations where at least one biometric can be obtained from part of the body.

In an embodiment, computational modeling algorithms are used to improve the accuracy of the sensors described above.

Common mode rejection can also be used to improve the accuracy of the sensors described above. In an embodiment, ECG signals are recorded using two different metal conductive pads on either contact point on opposing sides of the body. Using an ECG analog front end (AFE), a signal from the difference between the sum of each side's contact pads can be taken—this design may have noise problems. In 12 lead ECGs, many different channels are taken across sides of the body and the control channels are subtracted from the data channels to reduce electrical body noise. This strategy is more difficult with fewer contact points.

In order to attempt the same noise cancelling common mode rejection, a multi-channel (at least 2) ECG AFE can be used, as well as a setup with multiple electrical contact points going to the same extremity. In an embodiment with a 3 channel ECG AFE, a channel is used for the regular across the body electrical difference. After this the other 2 channels are used for each respective side of the body, from one electrical contact on one side of the body to the other one on the same side.

Because of Einthoven's triangle, there will be no electrical signal from the heart at these second and third channels of ECG. Therefore, any electrical signals that these channels pickup must be body or environmental noise and can safely be factored out of the data channel in order to get a cleaner signal.

In an embodiment, a method for factoring out this noise from our data channel is IFFT(FFT(ch_0)−(a*FFT(ch_1)+b*FFT(ch_2)). Where ch_0 is the data channel, and ch_1 and ch_2 are noise cancelling channels, a and b are scalar constants to decide how much noise cancellation can be done without accidentally getting rid of some signal.

An FFT form of these data channels can be used to cancel out data because it makes sense to cancel out the frequencies of noise that occur in the body more than the raw output of the data. In addition using this method it is possible to run bandpass filters on different channels to isolate specific noise before and after application of filters.

Measurements & Calculations

In an embodiment, the following measurements can be taken.

Inner beat Interval Data (IBI): This is the time interval between individual heartbeats. IBI data is used to form heart rate waves, which is defined as heart rate variability herein.

Heart Rate: This is beats per minute. Average resting or real-time BPM can be calculated.

Peak-valley difference: The RMS value from the ECG beat difference wave is used as the peak valley difference. In an embodiment, peak valley difference is calculated using a moving average of the absolute value of the change in peak to peak heart beat over each new beat. This value is normalized to a 0-1 scale by using the max of some constant and the best value this user has achieved. The optimal value is 0. The equation looks like the following, where:
PV=the scaled Peak Valley difference (based on the RMS value from the beat difference wave) or “HRV value”
v=the new peak-to-peak heartbeat difference data point
p=a constant for the moving average. In an embodiment, p is 40.
k=a constant to scale the output.
best=the maximum value of adjusted PV previously achieved by the person.

Peak Valley Difference can be based on actual or guided respiration, where the peak is measured at end of inhalation and the valley is measured at end of exhalation. This measurement is defined within as heart rate variability.

Frequency Domain Heart Rate Variability: A discrete Fourier transform is used to measure the power distribution across frequencies. Typically, dominant Low Frequency (LF) is desired when breathing slowly and dominant High Frequency (HF) is desired otherwise due to its relationship with parasympathetic and sympathetic nervous system activity. This measurement is defined as heart rate variability herein.

Heart Rhythm Smoothness: Smoothness is a measure of the heart rate waveform consistency. In an embodiment, this algorithm takes the heart rate wave and finds peaks and valleys. Peak to Peak is measured and Valley to Valley is measured. If n peak-peak/valley-valley measurements are within a threshold t of each other, the data is said to be smooth enough. (values that can be used are 4 for n and 1000 milliseconds for t). This measurement is defined as heart rate variability herein.

Time Domain Heart Rate Variability: These are time-domain measurements of heart rate variability. In an embodiment, RMSSD and STNN are calculated. In an embodiment, these measurements can be used to assess baselines in autonomic flexibility when the user is not breathing slowly.

Rate of change: The rate at which a user's metrics change can be an indication of how well the user transitions from a sympathetically dominant to parasympathetically dominant state. It's also an indicator of vagal tone. In an embodiment, the following equation can be used:

Sₙ=Pₙ+k*(Pₙ−P₀)/n

### Where:

P₀=the Score at time increment t=0 (typically the time increment immediately preceding n), before the rate of change is accounted for
Pₙ=the VT Score at time increment t=n, before the rate of change is accounted for
S=the final Score at time increment t=n.
k=a constant that weights how important Rate of Change is (e.g., previously at 0.2)

Respiration Correlation with The Simulated Respiration Wave: The simulated respiration wave is based on the desired (or “directed”) breathing rate. The Respiration Correlation value is a measure of how close the user is to the desired rate. The Respiration Correlation value is calculated according to this equation and runs between 0-1 with the optimal value being 0. In an embodiment, the following equation can be used:

$${C}_{t} = cos ( \frac{2 t π}{b} ) - {r}_{t}$$

Where:
Cₜ=the correlation of the actual person's breathing rate to a target breathing rate, or “Respiration Correlation value”
t=the time increment
b=the target breathing speed of the individual
r=the respiration value taken at time t

The respiration value (“r”) taken at time t is the peak adjusted respiration measurement, where “adjusted” means the data received from the respiration sensor is adjusted so that a complete inhalation results in a value of 1 and a complete exhalation results in a value of −1. In an embodiment, the respiration correlation value is used to adjust the any calculation involving a simulated respiration wave to take into account actual user respiration.

Respiratory Sinus Arrhythmia: Respiratory Sinus Arrhythmia, also known as synchrony and phase difference, is the inverse of phase angle between the sine wave calculated by heart rate IBI data, and a sine wave of actual or simulated respiration. An increase in synchrony corresponds to a movement towards an ideal breathing rate. In an embodiment, synchrony is calculated with the following equation:

(Heart rate wave peak−respiration wave peak)+(heart rate wave valley−respiration wave valley)/2;

Where each peak and valley is measured in milliseconds from a common starting point. Three of these calculations are averaged in each loop. Outlier data points are thrown away. In an embodiment, this measurement can be based on beat to beat blood pressure as well or instead of either heart rate or respiration. Blood pressure has an inverse phase relationship to respiration and heart rate. Respiratory sinus arrythmia can also be defined as phase angle herein.

Modulated Delta Train (MDT) Computational Model: Respiration modulates heartbeats. The spacing between two heartbeats changes during a respiration cycle. In other words, a plot of instantaneous heart rate against time shows a periodic signal having a frequency close to that of the respiration rate. This signal is called the Heart Rate wave (HR wave). It is observed that this signal contains vital information about the amount of oxygen being absorbed by the human body.

The heart and respiration rate are highly correlated. There is a one-to-one correspondence between a respiration and an HR wave cycle. A respiration cycle comprises an inhalation and an exhalation. The peak of the respiration cycle occurs when an inhalation is completed. The American Heart Association states that the normal (mean) resting adult human heartrate is between 60-100 beats per minute (BPM). The HR wave captures the deviation in the heartrate from a person's mean heartrate. It is observed that this deviation in the heartrate, within one respiration cycle, can be of the order of 5 to 15 BPM. The difference between the maximum and the minimum value of this heart rate will be referred to as PVD (Peak Valley Difference). The phase-difference is defined as the time difference between the peak of an HR wave and the corresponding peak of a respiration cycle in radians. The PVD and the phase-difference of the HR wave with respect to the corresponding respiration cycle are functions of the respiration rate. It has been observed that breathing at the respiration rate at which the amplitude of the HR wave is maximum, and the phase-difference with respect to the corresponding respiration cycle is minimum, provides many physiological and psychological benefits. These benefits include improved blood pressure, better athletic performance, and improvements in mood, pain management and anxiety management.

A prominent method of capturing heartbeats is the ECG signal. In an embodiment, the instantaneous heart rate can be measured by first detecting heartbeats (R peaks) in the ECG signal and then measuring the time difference between two consecutive beats. The reciprocal of this time difference times 60 gives an estimate of the instantaneous heartrate (HR wave) in BPM. As the HR wave measurement depends on the time difference between two consecutive beats, this measurement is highly sensitive to detection accuracy. Noise in the ECG signal acquisition can result in missing a heartbeat or detecting a noise spike as a heartbeat. Missing a beat will double the time interval between two consecutive beats which will result in a sudden dip in the HR wave. Similarly, detecting a noise spike as heartbeat between two beats will result in a spike in the HR wave. This will result in incorrect measurements of the PVD and phase-difference of the HR wave signal. Thus, HR wave measurements are highly sensitive to noise and detection accuracy of heartbeats. In order to mitigate this sensitivity problem a new Modulated Delta Train (MDT) model is proposed. MDT estimates an HR wave by observing a sequence of heartbeats. In an embodiment, this algorithm is used on a photoplethysmogram (PPG) sensor.

An HR wave is a result of the modulation of heartbeats by a respiration cycle. Hence the frequency of modulation is very close to that of the respiration cycle. Thus, the modulated heartbeats can be modeled by a periodic delta train modulated by a sinusoid having a frequency equal to that of the respiration cycle. MDT models the behavior of the heartbeats during a respiration cycle. The parameters of the MDT model can be used to estimate the PVD and phase-difference of the HR wave. B(t) defines a model of MDT:

B(t)=Eₙ₌₀ⁿ⁻¹δ(t−(nτ+ΔT+a sin(2πfᵣnτ+φ))),

Where: δ( ) is the delta function, τ is the mean period between heartbeats, ΔT represents the time delay between the peaks (or trough) of the respiration cycle and the first heartbeat detected after it, a is the amplitude of modulation and it captures the maximum deviation in the heart rate from a person's mean heart rate, f is the respiration frequency (Hz), φ is the phase-angle and N is the number of beats in a respiration cycle. In an embodiment, B(t) can be used to model MDT over multiple respiration cycles.

In an embodiment, the sine function in the MDT is replaced by a linear combination of sine and cosine functions and their higher-order harmonics. In an embodiment, the sine function in the MDT is replaced by a function constructed by a linear combination of polynomials. In an embodiment, the sine function in the MDT is replaced by a function constructed using splines. In the above embodiments the linear combinations and spline coefficients are parameters of the MDT model and are estimated while fitting the MDT model to the heartbeats.

In the model the respiration rate is assumed to be fairly stable and its frequency (f) is estimated r through measurements. In an embodiment, f is estimated while fitting the MDT model to the heart r beats. In an embodiment, f is simulated respiration or measured by a respiration sensor such as one that uses impedance pneumography.

The mean period between heart beats (τ) is estimated based on detected beats. In an embodiment, an algorithm like “A Real-Time QRS Detection Algorithm” by Jiapu Pan and Willis J. Tomkins, is used to detect beats from a noisy ECG signal.

There are three main parameters ΔT, a, φ, that have to be estimated to model the MDT B(t). The parameters are estimated based on heartbeats detected in one or more respiration cycles. Let the time indices of detected heartbeats be denoted by vn and the time shifts of the delta function in the MDT model be v̅n, where the sub-script n indicates the nth beat in the time interval under consideration. The parameters ΔT, a, φ are found such that v̅n closely mimics vn. A cost function C can be defined such that its value increases if the model deviates from the detected ECG beats.

C(ΔT,a,φ)=f(v,v̅)

where, v=[v0, v1 . . . vN−1] and v̅=[v̅0, v̅1 . . . v̅N−1]. In an embodiment, this cost function can be a sum of squared errors function,

C(Δ,a,φ)=Σₙ₌₀^{N-1}(vₙ−v̅ₙ)²,

Where: v̅=nτ+ΔT+a sin(2πfnτ+φ).

In an embodiment, if in the detected heartbeats, the duration between two consecutive peaks is larger than τ+upperbound(a) then it indicates that there are a few missing heartbeats and the time indices corresponding to the missing heartbeats can be neglected from the cost function. Similarly, if the duration between two consecutive detected peaks is less that τ−upperbound(a) then it indicates that it is noise and it can be discarded from the sequence of detected beats.

In case of the sum of squared errors cost function, given the value of φ, the optimal values of the two parameters a, ΔT can be estimated in closed form as

$$a = \frac{\begin{matrix}{\sum }_{n = 0}^{N - 1} ( ( ( {v}_{n} - {w}_{n} ) - \frac{1}{N} {\sum }_{q = 0}^{N - 1} {v}_{q} - q τ ) \\ ( sin ( 2 π {f}_{r} n τ + φ ) - \frac{1}{N} {\sum }_{q = 0}^{N - 1} sin ( 2 π {f}_{r} q τ + φ ) ) )\end{matrix}}{{\sum }_{n = 0}^{N - 1} {( sin ( 2 π {f}_{r} n τ + φ ) - \frac{1}{N} {\sum }_{q = 0}^{N - 1} sin ( 2 π {f}_{r} q τ + φ ) )}^{2}} .$$

The estimated value of a can be used to estimate AT as follows

$$Δ T = \frac{1}{N} {\sum }_{n = 0}^{n = N - 1} {v}_{n} - ( n τ + α sin ( 2 π {f}_{r} n τ + φ ) ) .$$

In an embodiment, the cost function can be evaluated for various values of φ∈[0, 2π) and the value where the cost function is minimum can be chosen as an estimate for φ. In an embodiment, numerical methods, e.g. false position method or bisection method can be used to estimate the value of φ.

The Peak Valley Difference (PVD) and phase-difference of the HR wave can be extracted from the MDT model. The maximum amplitude of the HR wave will occur if the heart beats are symmetrically placed around the angle 2πf nτ+φ=2π. Based on this observation, the maximum PVD value from r the model is

$$P V D = 4 a sin ( 2 π {f}_{r} \frac{τ}{2} ) ,$$

and the phase-angle between the peak of the HR wave and the peak of the respiration cycle is given by

phase difference=<φ−2πfᵣΔT,

Note that PVD is dependent only on the amplitude of modulation (a) and the phase-difference is dependent on ΔT and φ.

The MDT model avoids taking a difference between consecutive heartbeats to estimate the HR wave. Furthermore, the estimation method is robust against noisy beats and missing beats. If the instantaneous heart rate (HR wave) is not periodic then the model will not fit the detected heartbeats. In other words, the fitting error C(Δ, a, φ) will be large if the HR wave is not periodic. The fitting error can be used as a metric to evaluate the regularity of the HR wave and also to evaluate the correlation of the HR wave frequency with the respiration frequency. The regularity of the HR wave is a measure of the confidence that the phase-angle and PVD have been accurately measured. Since the HR wave does not instantaneously follow changes in respiration rate, it is advisable to wait until the HR wave is regular, before measuring the performance of an individual.

Respiration

In an embodiment, respiration can be estimated with a heart rate sensor by fitting the MDT to heart beats. In an embodiment, the MDT fitting can be used to improve the accuracy of a respiration sensor. In an embodiment, the phase difference can be calculated at different rates using the simulated respiration signal or a respiration sensor. The sign of phase difference is linear for breathing rates where respiratory sinus arrhythmia is present. Therefore, phase difference can be derived for numerous breathing rates after the sign of phase difference has been calculated for a few rates. Measuring the heart rate or beat to beat blood pressure waveform allows for derivation of respiration from the corresponding phase angle, i.e. passive measurements with a heart rate sensor.

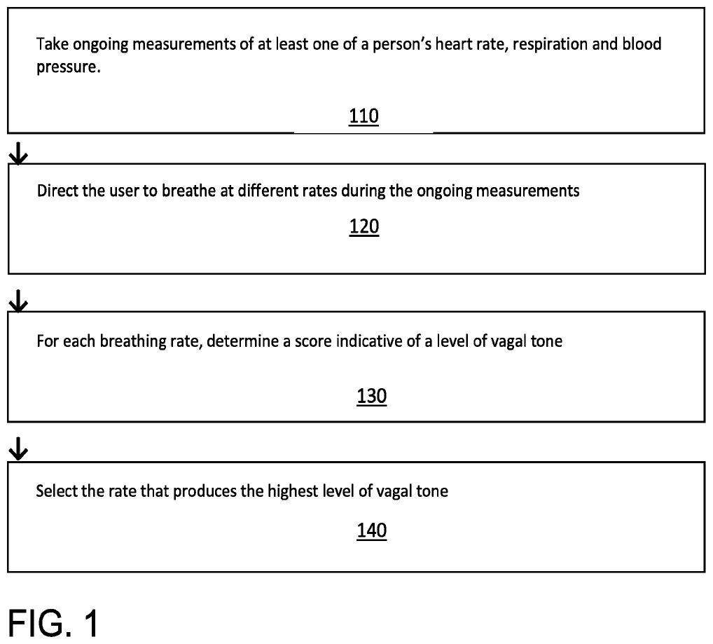

FIG. 1 is a flow chart of an embodiment of a first method for determining a Resonance Breathing Frequency. In FIG. 1, a method 100 may select a breathing rate that optimizes a user's vagal tone. To select a breathing rate that optimizes a user's vagal tone, in step 110, ongoing measurements of at least one of a user's heart rate, respiration, or blood pressure are taken. In step 120, the user breathes at different rates while measurements are taken. In step 130, a score is calculated for the level of vagal tone displayed. And in step 140, the rate that produces the highest level of vagal tone is selected. In an embodiment, the breathing rates selected in step 120, are based upon the previous vagal tone scores collected in step 130.

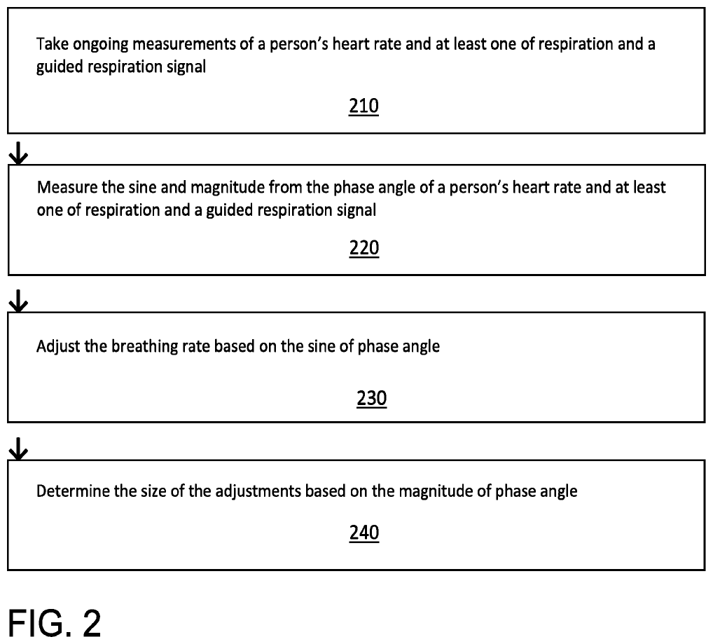

FIG. 2 is a flow chart of an embodiment of a second method for determining a Resonance Breathing Frequency. In FIG. 2, method 200 may select a breathing rate that optimizes a user's vagal tone. To select a breathing rate that optimizes a user's vagal tone, in step 210, ongoing measurements of a user's heart rate, and at least one of respiration and a guided respiration signal are taken. In step 220, the phase angle is calculated between the sine wave of a user's heart rate, and the sine wave of at least one of respiration and a guided respiration signal. In step 230, guided breathing rate is adjusted based on the calculated phase angle. In an embodiment, the sign of phase angle determines the direction of breathing rate adjustments in step 230. And in step 240, the size of guided breathing rate adjustment is based on the magnitude of the phase angle calculated above. In an embodiment, phase angle is calculated with the following equation:

(Heart rate wave peak−respiration wave peak)+(heart rate wave valley−respiration wave valley)/2;

Where each peak and valley is measured in milliseconds from a common starting point. Three of these calculations are averaged in each loop. Outlier data points are thrown away.

In an embodiment, smoothness is required to reach a threshold before adjustments are made. In an embodiment, adjustments are repeated until the confidence in best rate selection reaches a threshold, where the confidence adjustment algorithm is as follows:
Confidence starts at 0.

Confidence adjustment=0.1+((1−(magnitude/max magnitude)/k;
Where max magnitude is the largest change that we can measure.

In an embodiment, adjustments are based on the following algorithm:

Adjustment amount=(phase angle magnitude*(1−confidence))/k;

Where confidence of 1 is completely confident that we have found our correct value and confidence of 0 at start of process. K is a constant that we use to adjust the milliseconds of phase angle to adjustment amount of breaths/minute.

In an embodiment, a heart rate wave is constructed using an equation that fits all discrete points into a continuous equation for a sine wave. In an embodiment, respiration is measured with a respiration sensor, estimated from deriving phase angle at several rates, or constructed while fitting the MDT to heart beats.

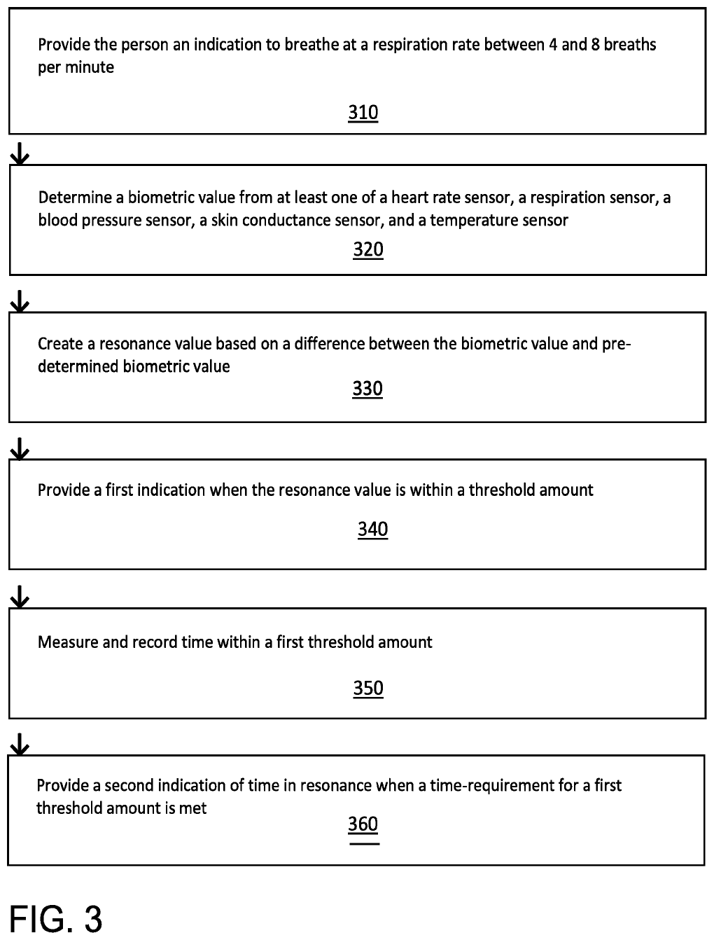

FIG. 3 is a description of an embodiment of a method for measuring and providing feedback on Resonance. Heart rate variability biofeedback benefits are received when individuals stay in Resonance for a sufficient period of time consecutively daily. Therefore, it's important to measure and indicate time in Resonance. In FIG. 3, a method 300 may indicate a time in resonance. To indicate resonance, in step 310, a user is given a breathing indicator at a rate between 4 and 8 breaths per minute. In step 320, at least one of heart rate, heart rate variability, skin conductance, skin temperature, respiration, and blood pressure are measured. In step 330, the differences between measured biometrics and pre-determined biometric values are calculated. In step 340, a first indication is provided when the resonance value is within a threshold amount, the indication being in the form of visual display, haptic feedback or auditory. In step 350, the time within a first threshold amount is measured. And in step 360, a second indication is provided when a time requirement for a first threshold amount is met, the indication being in the form of visual display, haptic feedback or auditory. In an embodiment, the predetermined value in step 330 may be based on previous data collected by the same user. In an embodiment, the historical measurement is the best recorded historical measurement. In an embodiment, the predetermined value in step 330 is 0-degrees phase difference.

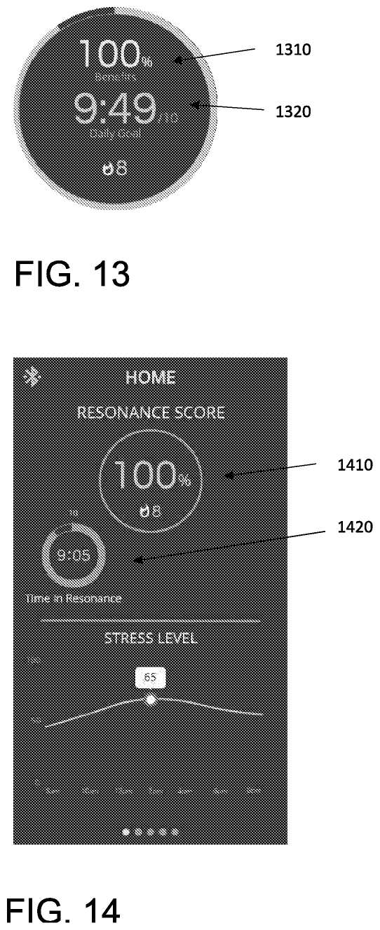

FIG. 13 is an embodiment of an exemplary visual display of the quantity of heart rate variability benefits received and time in resonance. In an embodiment, step 1320 indicates the time in resonance during a session, a day or a different duration using method 300. In an embodiment, the visual display is software on a smartwatch readily available in the market or a custom device.

FIG. 14 is an embodiment of an exemplary visual display of the quantity of heart rate variability benefits received and time in resonance. In an embodiment, step 1420 indicates the time in resonance during a session, a day or a different duration using method 300. In an embodiment, the visual display is software on a smartphone readily available in the market.

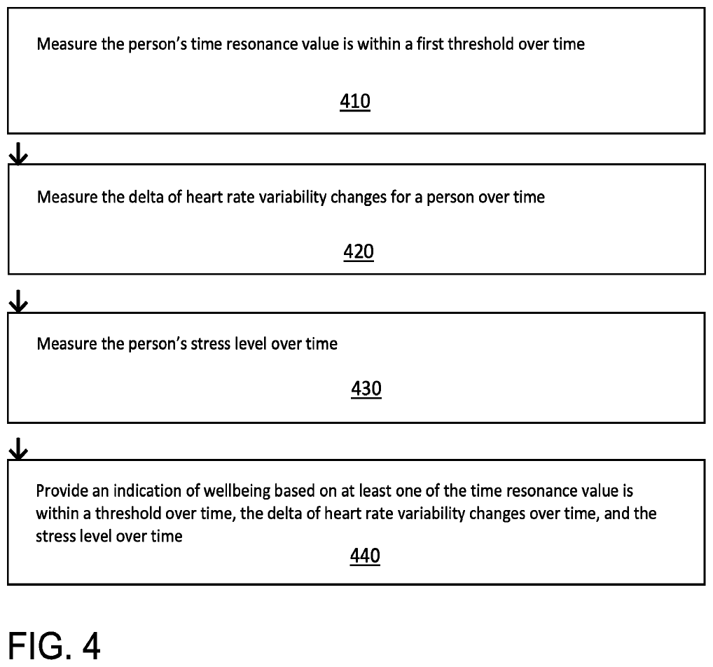

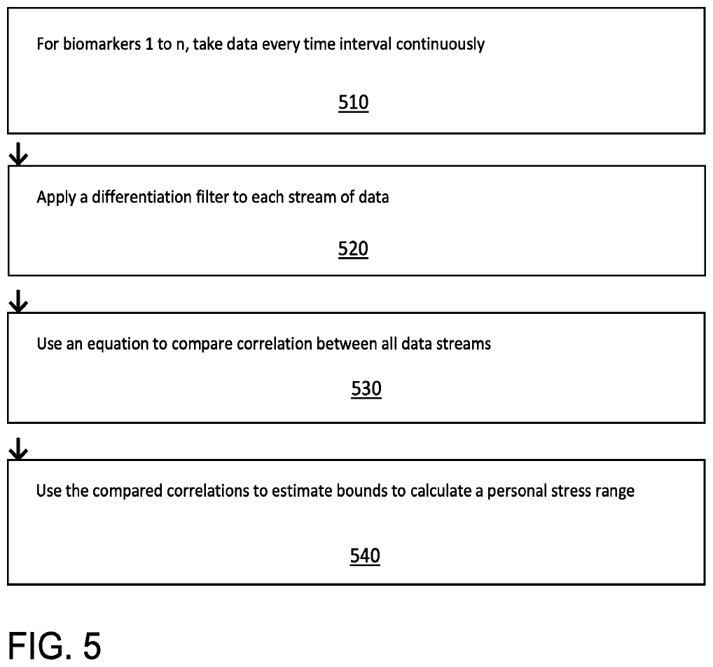

FIG. 4 is a description of an embodiment of methods for measuring and providing feedback on the quantity of Heart Rate Variability Biofeedback benefits received. In FIG. 4, a method 400 provides an indication of wellbeing. To provide an indication of wellbeing, in step 410, a user's time in resonance is measured. In step 420, a user's heart rate variability over time is measured. In step 430, a user's stress level over time is measured. And in step 440, an indication of wellbeing is provided based on at least one of the user's time in resonance, the user's heart rate variability over time, and the user's stress level over time. In an embodiment, this indication represents a quantity of heart rate variability biofeedback benefits received. In an embodiment, a user's stress level over time may be calculated using the method described in FIG. 5. In an embodiment, the data points measured over time may be calculated using a moving average. In an embodiment, periods of data are weighted differently in the moving average. In an embodiment, the user's heart rate variability over time is measured based on the delta of heart rate variability changes during slow breathing or exercise. In an embodiment, the delta is calculated using a frequentist approximation for a set's maximal element using the German tank problem. In an embodiment, this equation is N=m+(m/k)−1 where m is the max element in the set, k is the number of elements in the set, and N is an estimate for the max number of the given set with a linear distribution and random ordering.

FIG. 13 is an embodiment of an exemplary visual display of the quantity of heart rate variability benefits received and time in resonance. In an embodiment, step 1310 indicates the quantity of benefits received using method 400. In an embodiment, the visual display is software on a smartwatch readily available in the market or a custom device.

FIG. 14 is an embodiment of an exemplary visual display of the quantity of heart rate variability benefits received and time in resonance. In an embodiment, step 1410 indicates the quantity of benefits received using method 400. In an embodiment, the visual display is software on a smartphone readily available in the market.

FIG. 5 is a description of an embodiment of a first method for analyzing user stress level. Most metrics described herein are in some way correlated to stress level. As a person gets more stressed, skin temperature raises, skin conductivity lowers, HRV lowers, HR raises, and blood pressure elevates. All of these metrics have confounding factors, but given an analysis of all of them together, a stress metric can be derived. For example, when a person walks outside and it is a cold day, skin temperature could go down, but stress has not changed. Also increase in humidity could change skin conductance, etc. Beyond this each person has different natural ranges for these metrics. Two different people's skin temperature in a calm state could be very different. Measuring the correlation of the change in these metrics allows for a more precise estimate of a person's stress response. In an embodiment, these stress response data points are also used to calculate upper and lower bounds for an individual's stress response.

In FIG. 5 a method 500 may determine the level of stress within a personalized stress range for a user. To determine a user's level of stress within a personalized stress range, in step 510, data is collected from each of at least 1 available sensor at each time interval. In step 520, a differentiation filter is used on each individual stream of data collected. In step 530, An equation is used to compare all of the data streams to each other. And in step 540, the compared correlations are used to estimate upper and lower bounds of a user's personal stress range. In an embodiment a multidimensional covariance algorithm is used for step 530. In an embodiment, step 540 uses a frequentist solution to the German Tank Problem to calculate upper and lower bounds of a user's personal stress range. In this embodiment, the following equation can be used:

S_min=(1−1/n)*S_smallest,

S_max=(1+1/n)*S_biggest
Where: S_min is the lower bound of a user's personal stress range, S_max is the upper board of a user's personal stress range, n is the number of data points collected, S_smallest is the smallest data point collected, and S_biggest is the largest data point collected.

In an embodiment, method 500 is used to measure physical or psychological exertion for a user over a set amount of time.

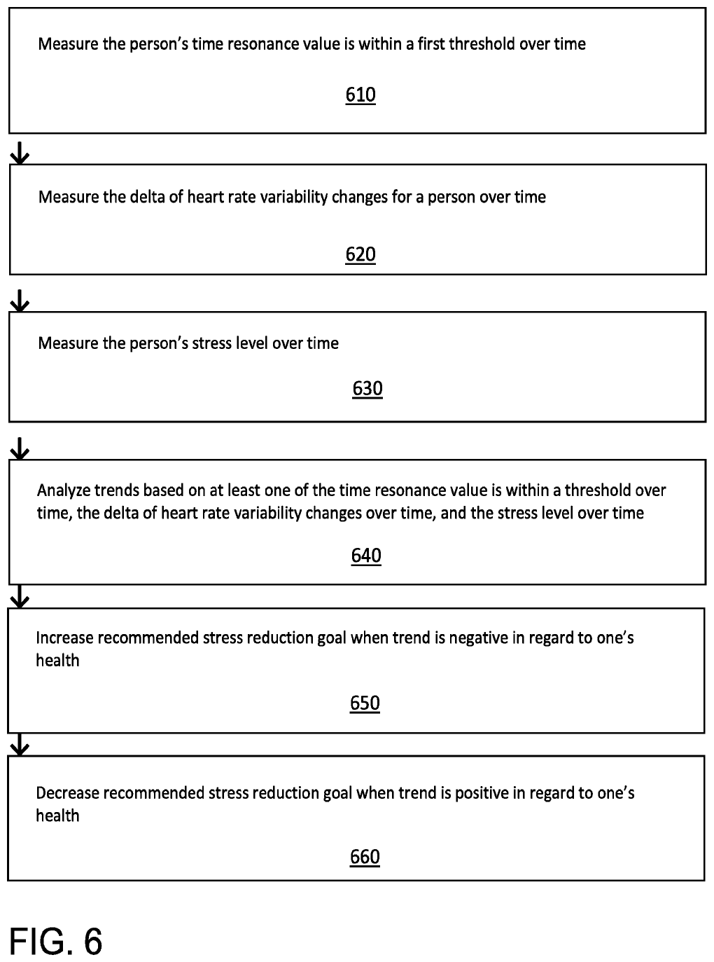

FIG. 6 is a description of an embodiment of a method for providing a personalized therapy regimen. In FIG. 6, a method may calculate a recommended stress reduction goal. To calculate a recommended stress reduction goal, in step 610, a user's time in resonance is measured and compared to a first threshold value. In step 620, the change in a user's heart rate variability is measured. In step 630, the user's stress level over time is calculated. In step 640, the trends based on at least one of the metrics calculated in 610, 620, and 630 are analyzed. In step 650, the recommended stress reduction goal is decreased if the trend is negative in regards to one's health. And in step 660, the recommended stress reduction goal is increased if the trend is positive in regards to one's health. In an embodiment, stress level in step 630 is calculated using method 500. In an embodiment, recommended stress reduction goal may be an analog for time in resonance, desired stress level baseline, athletic performance goal, or other relaxation means.

FIG. 13 is an embodiment of an exemplary visual display of the quantity of heart rate variability benefits received and time in resonance. In an embodiment, step 1320 indicates the time in resonance during a session, a day or a different duration and is modified using method 600. In an embodiment, the visual display is software on a smartwatch readily available in the market or a custom device.

FIG. 14 is an embodiment of an exemplary visual display of the quantity of heart rate variability benefits received and time in resonance. In an embodiment, step 1420 indicates the time in resonance during a session, a day or a different duration and is modified using method 600. In an embodiment, the visual display is software on a smartphone readily available in the market.

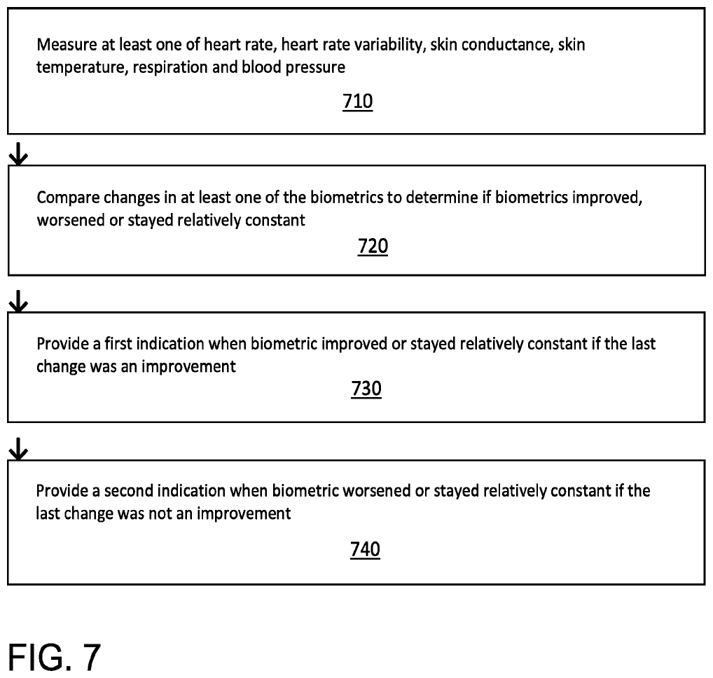

FIG. 7 is a description of an embodiment of a second method for analyzing user stress level and providing feedback. In FIG. 7, a method 700 may improve biometrics using biofeedback. To improve biometrics using biofeedback, in step 710, at least one of heart rate, heart rate variability, skin conductance, skin temperature, respiration, and blood pressure are measured. In step 720, the changes in at least one biometric are compared. In step 730, a first indication is provided when the biometric data improved or stayed constant after a previous improvement. And in step 740, a second indication is provided when the biometric data worsened or stayed constant after a previous worsening. In an embodiment, compared intervals may provide feedback on relaxation, stress reduction, mindfulness, or progress towards a desired athletic performance state. In an embodiment, operant conditioning and positive reinforcement are used within method 700.

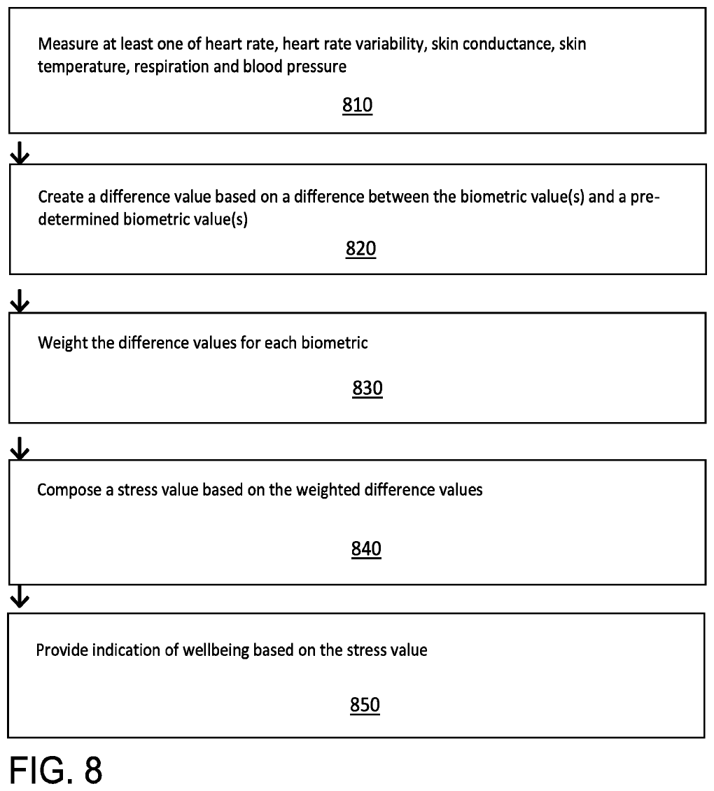

FIG. 8 is an exemplary block diagram of an embodiment of a method 800 for indicating wellbeing based on a measurement of stress level. To indicate wellbeing based on a measurement of stress level, in step 810, at least one of heart rate, heart rate variability, skin conductance, skin temperature, respiration, and blood pressure are measured. In step 820, the differences between measured biometrics and pre-determined biometric values are calculated. In step 830, each difference has a unique weight applied to it. In step 840, a single stress value metric is made by the combination of each weighted difference. And in step 850, an indication of wellbeing is provided based on the calculated stress value metric. In an embodiment, pre-determined biometric values may be based on previous data collected by the same user. In an embodiment, unique weights applied to each difference may be multiplied to each difference and may be based on the results of data from many different users. In an embodiment, the single stress value metric may be calculated with the sum of each of the weighted differences.

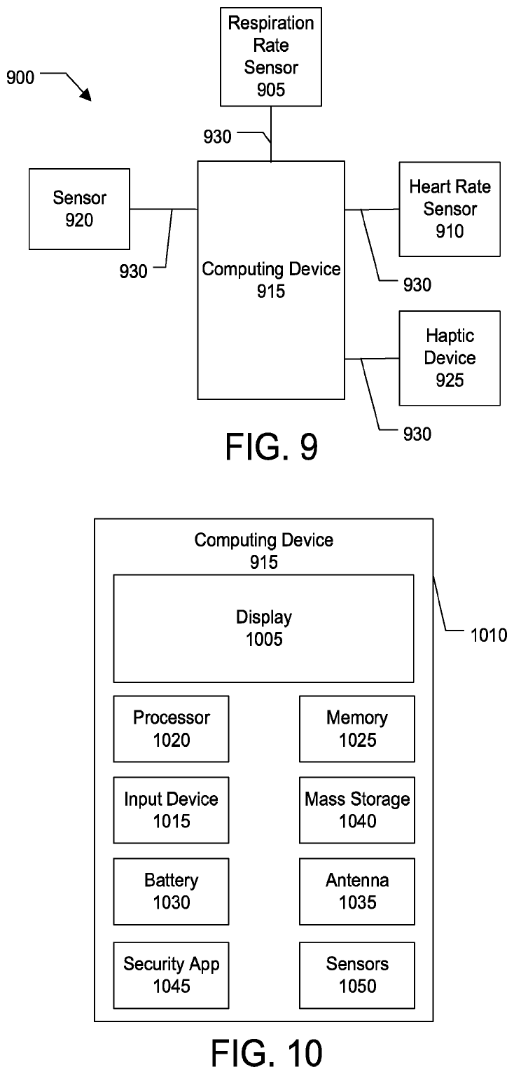

FIG. 9 is a simplified, exemplary block diagram of an embodiment of a system 900 for implementing the embodiments of systems and methods disclosed herein. System 900 may include a number of sensors, e.g., a respiration rate sensor 905 (e.g., as described within this disclosure) and a heart rate sensor 910 (e.g., as described within this disclosure), for developing data regarding a user. Sensors 905, 910, and 920 are in communication with a computing device 915. Computing device 915 may further be in control of a haptic device 925 and a buzzer or speaker (not shown) for communicating with the user. System 900 may be referred to as a Biometric Analysis Device.

Respiration rate sensor 905 may be an impedance-based sensor as discussed within this specification. Heart rate sensor 910 may be, e.g., a plurality of sensors sufficient to produce an electrocardiogram (ECG, as discussed within), a chest-mounted device, or a wrist-mounted device, so long as the device provides heart rate data with sufficient accuracy and precision. Sensor 920 is representative of additional sensors that may be included, such as sensors for determining galvanic skin response, temperature, blood pressure, hydration, sleep, exercise activity, brain activity, nutrient levels, or blood analysis. Sensors 905, 910, and 920 may supply data to computing device 915 via communication links 930.

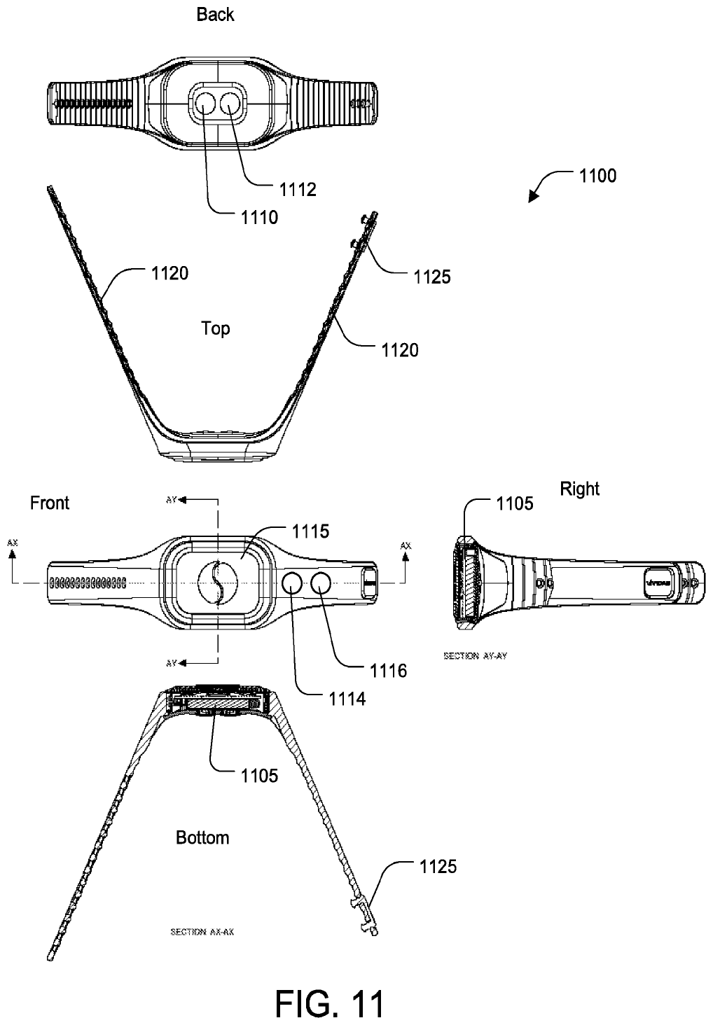

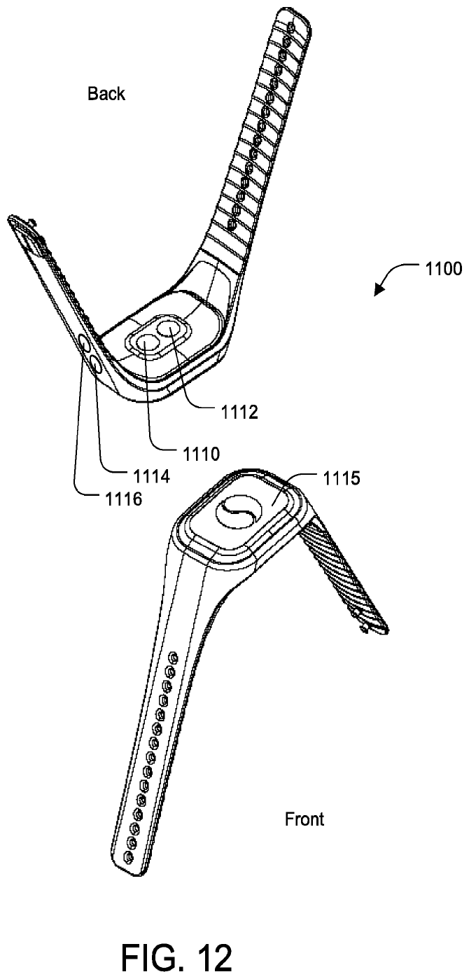

Computing device 915 may include a user interface and software, which may implement the steps of the methods disclosed within. Computing device 915 may receive data from sensors 905, 910, and 920, via communication links 930, which may be hardwire links, optical links, satellite or other wireless communications links, wave propagation links, or any other mechanisms for communication of information. Various communication protocols may be used to facilitate communication between the various components shown in FIG. 9. Distributed system 900 in FIG. 9 is merely illustrative of an embodiment and does not limit the scope of the systems and methods as recited in the claims. In an embodiment, the elements of system 900 are incorporated into a single, wearable Biometric Analysis Device (e.g., as described regarding FIGS. 11 and 12). One of ordinary skill in the art would recognize other variations, modifications, and alternatives. For example, more than one computing device 915 may be employed. As another example, sensors 905, 910, and 920 may be coupled to computing device 915 via a communication network (not shown) or via some other server system.

Computing device 915 may be responsible for receiving data from sensors 905, 910, and 920, performing processing required to implement the steps of the methods, and for interfacing with the user. In some embodiments, computing device 915 may receive processed data from sensors 905, 910, and 920. In some embodiments, the processing required is performed by computing device 915. In such embodiments, computing device 915 runs an application for receiving user data, performing the steps of the method, and interacting with the user. In other embodiments, computing device 915 may be in communication with a server, which performs the required processing, with computing device 915 being an intermediary in communications between the user and the processing server.

System 900 enables users to access and query information developed by the disclosed methods. Some example computing devices 915 include desktop computers, portable electronic devices (e.g., mobile communication devices, smartphones, tablet computers, laptops) such as the Samsung Galaxy Tab®, Google Nexus devices, Amazon Kindle®, Kindle Fire®, Apple iPhone®, the Apple iPad®, Microsoft Surface®, the Palm Pre™, or any device running the Apple iOS®, Android® OS, Google Chrome® OS, Symbian OS®, Windows Mobile® OS, Windows Phone, BlackBerry® OS, Embedded Linux, Tizen, Sailfish, webOS, Palm OS® or Palm Web OS®; or wearable devices such as smart watches, smart fitness or medical bands, and smart glasses.

FIG. 10 is an exemplary block diagram of a computing device 915 from the system of FIG. 9. In an embodiment, a user interfaces with the system through computing device 915, which also receives data and performs the computational steps of the embodiments. Computing device 915 may include a display, screen, or monitor 1005, housing 1010, input device 1015, sensors 1050, and a security application 1045. Housing 1010 houses familiar computer components, some of which are not shown, such as a processor 1020, memory 1025, battery 1030, speaker, transceiver, antenna 1035, microphone, ports, jacks, connectors, camera, input/output (I/O) controller, display adapter, network interface, mass storage devices 1040, and the like. In an embodiment, sensors 1050 may include sensors 905, 910, and 920 incorporated into computing device 915, and haptic device 925 may also be incorporated into device 915. In an embodiment, housing 1010 is the housing of the wearable biometric analysis device 1000 of FIGS. 10 and 11.

Input device 1015 may also include a touchscreen (e.g., resistive, surface acoustic wave, capacitive sensing, infrared, optical imaging, dispersive signal, or acoustic pulse recognition), keyboard (e.g., electronic keyboard or physical keyboard), buttons, switches, stylus, or combinations of these.

Display 1004 may include dedicated LEDs for providing directing signals and feedback to a user.

Mass storage devices 1040 may include flash and other nonvolatile solid-state storage or solid-state drive (SSD), such as a flash drive, flash memory, or USB flash drive. Other examples of mass storage include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), battery-backed-up volatile memory, tape storage, reader, and other similar media, and combinations of these.

System 900 may also be used with computer systems having configurations that are different from computing device 915, e.g., with additional or fewer subsystems. For example, a computer system could include more than one processor (i.e., a multiprocessor system, which may permit parallel processing of information) or a system may include a cache memory. The computing device 915 shown in FIG. 10 is but an example of a computer system suitable for use. For example, in a specific implementation, computing device 915 is a wrist-mounted Biometric Analysis Device in communication with or incorporating the sensors of FIG. 10. An example of such a Biometric Analysis Device is discussed regarding device 1000 of FIGS. 10 and 11. Other configurations of subsystems suitable for use will be readily apparent to one of ordinary skill in the art. In other specific implementations, computing device 915 is a mobile communication device such as a smartphone or tablet computer. Some specific examples of smartphones include the Droid Incredible and Google Nexus One®, provided by HTC Corporation, the iPhone® or iPad®, both provided by Apple, BlackBerry Z10 provided by BlackBerry (formerly Research In Motion), and many others. The Biometric Analysis Device may be a laptop or a netbook. In another specific implementation, the Biometric Analysis Device is a non-portable computing device such as a desktop computer or workstation.

In an embodiment, system 900 may be incorporated into a single module. The module may have four user contacts (or “electrodes”) placed to allow a user to make contact with two contacts with one user extremity and with the other two contacts with the other user extremity. This module can be contained within numerous types of wristband straps (leather, etc.) and form factors (such as key chain, steering wheel cover, etc.). The module, or the strap or other form factor, may also include a small OLED display to display the current time. The module may execute software that performs an embodiment of the method. Accordingly, the module may provide the user with feedback, e.g., an indication of the user's respiration rate or heart rate or both.

FIG. 11 includes front, back, top, bottom, and right views of an embodiment of a wearable biometric analysis device 1100 for implementing embodiments of the methods disclosed within. Components and capabilities of biometric analysis device 1100 are also described with reference to FIGS. 2-10. Biometric Analysis Device 1100 includes a computing device 1105 and a sensor coupled to electrical contacts 1110, 1112, 1114, 1116 that acquire data that may be used to provide a measure of the user's respiration rate as discussed above. Computing device 1105 processes biometric data measured by the sensor(s) and produces feedback correlating to the processed biometric data. By continuously monitoring one or more biometric values, the user may respond to the data received and modify their behavior or activity to improve health and performance. The biometric analysis device 1100 thereby provides feedback by sensing and reporting a biometric value measured by the sensor to the user in real time. In an embodiment, contacts 1110, 1112, 1114, 1116 provide data to a TI ADS1292R sensor. As such, Biometric Analysis Device 1100 may be equipped with both a respiration rate sensor and a heart rate sensor. Computing device 1105 is in communication with the sensor or sensors associated with contacts 1110, 1112, 1114, 1116. Computing device 1105 may also control a haptic device (not shown) for communicating with the user. Computing device 1105 may include a display 1115, a user interface, and software, for implementing the steps of the methods disclosed within. In an embodiment, contacts 1110 and 1112 may correspond to contacts 202 and 302 as described above, and contacts 1114 and 1116 may correspond to contacts 204 and 304 as described above. In the embodiment, a method for determining the user's respiration rate includes the user placing device 1100 on one of the user's wrists such that contacts 1110, 1112 are in contact with the user's wrist. Then, the user brings contacts 1114, 1116 in contact with another part of the user's body such as one or more fingers on the user's opposing hand. In other words, the user touches contacts 1114, 1116 to a part of the user's body so that some or all of the user's chest is between contact pairs 1110, 1112 and 1114, 1116 (the circuits are described with reference to FIGS. 2 and 3 and contact pairs 202, 302 and 204, 304). In an embodiment, the part of the user's body may be a finger or other part of the opposing arm, may be a section of the user's torso, or may be a section of a leg of the user. With both contact pairs 1110, 1112 and 1114, 1116 in such contact with the user, the device then determines the user's respiration rate, heart rate, or both according to the methods described within.

Computing device 1105 may receive data from sensors 1110, 1112, 1114, 1116, perform processing required to implement the steps of the methods disclosed within, and provide a user interface via display 1115. In some embodiments, all processing required is performed by computing device 1105. In such embodiments, computing device 1105 executes instructions for receiving user data, performing the steps of the method, and interacting with the user. In other embodiments, computing device 1105 may be in communication with a server, which performs part of the required processing, with computing device 1105 being an intermediary in communications between the user and the processing server.

As illustrated, Biometric Analysis Device 1100 generally comprises a band 1120 configured to be worn about a wrist of the user. The band 1120 includes an adjustment mechanism 1125, for adjusting a circumference of the band 1120. A user can thus select, using adjustment mechanism 1125, a particular size for positioning band 1120 about the user's wrist. A visual indication, e.g., for feedback, may be provided by display 1115. In an embodiment, visual indicators may be further be positioned on the band 1120 to provide visual signals to the user. Sensor(s) associated with contacts 1110, 1112, 1114, 1116 may be configured to be activated by computing device 1105. In an embodiment, additional sensors, e.g., a temperature sensor or a galvanic response sensor, may be provided to provide more user data for determining vagal tone. In an embodiment, one or more translucent windows may be positioned about the band 1120 to transmit light from one or more indicators positioned with the band 1120.

Biometric analysis device 1100, in one embodiment, is used measure a user's respiration rate. Accordingly, the biometric analysis device 1100 may provide the user with real-time, personal biofeedback. In an embodiment, device 1100 may measure both a user's respiration rate and heart rate and provide feedback regarding one or both. The biofeedback may allow the user to learn about the user's personal physiological state and physiological responses. As a result, the biofeedback provided to the user (by, e.g., one or more of display 1115, or haptic device, or speaker) may enable the user to self-regulate the user's activity and behavior to improve the user's performance or health. In an embodiment, device 1100 may provide a user with feedback (e.g., a vibration pattern of frequency, duration, and magnitude) selected to encourage a desired behavior. In an embodiment, biometric analysis device 1100 is configured to provide the user with feedback with reference to previously-collected biometric data, such as respiration rate or heart rate variability. The biometric analysis device 1100 may emit vibrations based on the user's actual respiration rate, or a target respiration rate. For example, a visual indication from, e.g., display 1115, may be provided and configured to emit different colors based on when the user is supposed to inhale and exhale for deep breathing relaxation techniques. The user may also be capable of changing the breathing intervals. The visual indication and breathing intervals may be enabled and adjusted through the user interface.

FIG. 12 is a perspective view of the biometric analysis device of FIG. 11.

FIGS. 11 and 12 illustrate one example embodiment of a wearable biometric analysis device 1100 that is configured to measure the respiration rate of a user. In one embodiment, biometric analysis device 1100 can include each of the elements of system 900 of FIG. 9 and FIG. 10. In other embodiments, biometric analysis device 1100 can include other elements that function with biometric analysis device 1100 to provide biometric measurement and analysis to assist a user with stress management.

The following paragraphs include enumerated embodiments.

1. An embodiment of a method for Resonance Frequency with two HRV in VT score where it cycles based on predetermined rates. The method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, a first heart rate variability value for each of the first plurality of time periods; determining, from the heart rate measurements taken of the user a second heart rate variability value for each of the first plurality of time periods; combining in whole or in part, for each of the first plurality of time periods, the first heart rate variability value and the second heart rate variability value to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

2. An embodiment of a method for providing Resonance Frequency with HRV and RSA guided. The method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, a first heart rate variability value for each of the first plurality of time periods; determining, from the heart rate measurements taken of the user, a first respiratory sinus arrythmia value for each of the first plurality of time periods; combining in whole or in part, for each of the first plurality of time periods, the first heart rate variability value and the first respiratory sinus arrhythmia value to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

3. An embodiment of a method for providing Resonance Frequency with at least one HRV measurement and cycling based on historical measurements. The method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, at least one heart rate variability value for each of the first plurality of time periods; combining in whole or in part, for each of the first plurality of time periods, the at least one heart rate variability values to create a first vagal tone value for each of the first plurality of time periods; determining the pre-determined respiration frequencies provided using first vagal tone values; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

4. An embodiment of a method for providing Resonance Frequency with phase angle score. The method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a heart rate sensor, taking first heart rate measurements of the user for each of the first plurality of time periods; using a respiration sensor or the guided respiration signal, taking first respiration measurements of the user for each of the first plurality of time periods; determining, from the heart rate measurements and the respiration measurements, a first phase angle value for each of the first plurality of time periods; combining in whole or in part, for each of the first plurality of time periods, the first heart rate variability values to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

5. An embodiment of a method for providing a Resonance Frequency with phase angle adjustments. The method comprising: using a heart rate sensor, taking first heart rate measurements of the user; using a respiration sensor or the guided respiration signal, taking first respiration measurements of the user; determining, from the heart rate measurements and the respiration measurements, a first phase angle value for each of the first plurality of time periods; providing the user with an indication to breathe at respiration frequencies, adjusting the indicated respiration frequency based on the sign of the first phase angle value; and stopping the adjusting of the indicated respiration frequency when sufficiently confident that the indicated respiration frequency is the best respiration frequency for the user.

6. The embodiment 5, wherein the confidence in the indicated respiration frequency being best is based on 0 degrees phase angle.

7. An embodiment of a method for providing Resonance measurement and feedback. The method comprising: initiating a feedback session by user action or software prompt; providing the user with an indication to breathe at a calculated respiratory frequency, the rate being between 4 and 8 breaths per minute; determining a biometric value from at least one of: a heart rate sensor and a respiration sensor, the biometric value(s) being heart rate variability, phase angle or respiration accuracy; creating a vagal tone value based on a difference between the biometric value and a pre-determined biometric value; providing, to the user, a first indication of vagal tone when the vagal tone value is within a first threshold amount, the first indication of vagal tone provided using a first signal from one of: a visual interface, an audio interface, and a haptic interface; measuring and recording time within a first threshold amount for each session; and providing, to the user, a second indication of vagal tone when a time-requirement for a first threshold amount is met, the second indication of vagal tone provided using a second signal from one of: a visual interface, an audio interface, and a haptic interface.

8. The embodiment 7, wherein the pre-determined value is the best recorded value from past sessions or 0 degree phase angle.

9. An embodiment of a method for providing a Resonance score. The method comprising: using a heart rate sensor, analyzing heart rate variability during slow breathing; comparing the delta of HRV measurements across sessions; estimating the distance of current HRV from HRV at lowest attainable delta; and providing an indication of the distance, the indication being in the form of a visual display, haptic motor or auditory.

10. The embodiment 9, the method further comprising weighting the average calculated in embodiment 12 with this measurement for one indication based on at least two metrics.

11. The embodiment 9, the method further comprising weighting stress analysis with this measurement.

12. An embodiment of a method for providing a Resonance score. The method comprising: using a heart rate sensor, analyzing at least one of heart rate variability and phase angle during slow breathing, where the breath rates are between 4 and 8 breaths per minute; measuring the time the at least one biometric is within a first threshold across sessions; averaging the time the at least one biometric is within a first threshold across sessions; and providing an indication of the time that at least one biometric is within a first threshold across sessions, the indication being in the form of a visual display, haptic motor or auditory.

13. The embodiment 12, the method further comprising weighting the difference in delta in embodiment 9 with this measurement for one indication based on at least two metrics.

14. The embodiment 12, the method further comprising weighting stress analysis with this measurement.

15. An embodiment of a method for analyzing a stress level of a user. The method comprising: using at least one biometric sensor, taking at least two biometric measurements of the user; applying a differentiation filter to each type of biometric data; comparing correlations between all types of biometric data; and estimating lower and upper bounds of user stress level from the compared correlations using a linear form of variance.

16. The embodiment 15, wherein the correlations are calculated using a multidimensional covariance algorithm.

17. The embodiment 15, wherein correlations are based on analyzing first derivative changes in metrics.

18. The embodiment 15, wherein the lower and upper bounds are calculated using a solution to the German tank problem.

19. An embodiment of a method for providing a Therapy regimen. The method comprising: using a heart rate sensor, analyzing heart rate variability during slow breathing; comparing the delta of HRV measurements across sessions; estimating the distance of current HRV from HRV at lowest attainable delta; measuring changes in the estimated distance across sessions; analyzing trends in the measured changes in the estimated distance across sessions; and increasing recommended stress reduction goal when trends is negative in regard to health.

20. The embodiment 19, the method further comprising: decreasing recommended stress reduction goal when trend is positive in regard to health.

21. The embodiment 19, the method further comprising: weight the average calculated in claim 24 with this measurement to analyze trends based on at least two metrics.

22. The embodiment 19, the method further comprising: weight stress level in analysis of trends.

23. The embodiment 19, the method further comprising: stress reduction goal being analog for time in resonance, desired stress level baseline, athletic performance goal, or other relaxation means.

24. An embodiment of a method for providing a Therapy regimen. The method comprising: using a heart rate sensor, analyzing at least one of heart rate variability and phase angle during slow breathing, where the breath rates are between 4 and 8 breaths per minute; measuring the time the at least one biometric is within a first threshold across sessions; averaging the time the at least one biometric is within a first threshold across sessions; measuring changes in the average time the at least one biometric is within a first threshold across sessions; analyzing trends in changes in the average time the at least one biometric is within a first threshold across sessions; and increasing recommended stress reduction goal when trends is negative in regard to health;

25. The embodiment 24, the method further comprising: decreasing recommended stress reduction goal when trend is positive in regard to health.

26. The embodiment 24, the method further comprising: weight the difference in delta calculated in claim 19 with this measurement to analyze trends based on at least two metrics.

27. The embodiment 24, the method further comprising: weight stress level in analysis of trends.

28. The embodiment 24, the method further comprising: stress reduction goal being analog for time in resonance, desired stress level baseline, athletic performance goal, or other relaxation means.

29. An embodiment of a system for providing Stress feedback. The system comprising: a plurality of sensors configured to collect biometric data from the user; at least one processor having memory and instructions, the at least one processor being coupled to the plurality of sensors and configured to receive the biometric data from the plurality of sensors; a first indicator coupled to the at least one processor and activated by a control signal from the at least one processor, and the instructions, when executed by the at least one processor while the device is being worn by the user, causing the at least one processor to: receive first biometric data from the plurality of sensors throughout a first time interval; calculate at least one biometric value from all the received first biometric data, the biometric value being at least one heart rate, heart rate variability, respiration, blood pressure, skin conductance, and skin temperature; receive second biometric data from the plurality of sensors throughout a second time interval immediately following the first time interval; calculate the second biometric value from all the received second biometric data; compare the second biometric value to the first biometric value and when a result of the comparison is that the second biometric value is increased over the first biometric value by a threshold amount activate the first indicator to indicate an improvement of the at least one second biometric value from the at least one first biometric value; receive third biometric data from the plurality of sensors throughout a third time interval immediately following the second time interval; calculate the third biometric value from all the received third biometric data; compare the third biometric value to the second biometric value and when a result of the comparison is that the third biometric value is increased over the second biometric value by the threshold amount or the third biometric value stayed within a threshold amount from the second biometric value as long as the comparison from the first and second biometric value resulted in a first indication, activate the first indicator to indicate an improvement of the third biometric value from the second biometric value; consider the third biometric value to be a reference biometric value and the third time interval to be a reference time interval; after receiving the third biometric data, receive new biometric data from the plurality of sensors throughout a new time interval immediately following the reference time interval; calculate a new biometric value from all the received new biometric data; compare the new biometric value to the reference biometric value and when the result of the comparison is that the new biometric value is increased over the reference biometric value by the threshold amount or the new biometric value stayed within a threshold amount from the reference biometric value as long as the comparison from the third biometric value and second biometric values resulted in a first indication, activate the first indicator to indicate an improvement of the new biometric value from the reference HRV value; select the at least one new biometric value as the reference biometric value and the new time interval as the reference time interval; and repeat the receiving of new biometric data, the calculating of new biometric value, and the comparing of the new biometric value to the reference biometric value until it's desirable to end the repeating of indicating comparisons in biometric data.

30. An embodiment of a method for providing Stress feedback. The method comprising: using at least one sensor, measuring at least one biometric value, the biometric value being at least one of heart rate, heart rate variability, skin conductance, skin temperature, blood pressure and respiration; measuring the differences between the measured biometric values and predetermined biometric values; applying a weight to each measured difference; combining the weighted differences in a single metric; and providing an indication of wellbeing based on the single metric composed of weighted differences.

31. The embodiment 30, wherein the pre-determined biometric values are the best recorded measurements from historical data collection.

32. The embodiment 30, wherein the weights can adjust based on the difference between measured biometric values and predetermined biometrics reaching pre-determined thresholds or 0 degrees phase difference.

In the description above and throughout, numerous specific details are set forth in order to provide a thorough understanding of an embodiment of this disclosure. It will be evident, however, to one of ordinary skill in the art, that an embodiment may be practiced without these specific details. In other instances, well-known structures and devices are shown in block diagram form to facilitate explanation. The description of the preferred embodiments is not intended to limit the scope of the claims appended hereto. Further, in the methods disclosed herein, various steps are disclosed illustrating some of the functions of an embodiment. These steps are merely examples and are not meant to be limiting in any way. Other steps and functions may be contemplated without departing from this disclosure or the scope of an embodiment.

We claim:

1. A method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, a first heart rate variability value for each of the first plurality of time periods; determining, from the heart rate measurements taken of the user a second heart rate variability value for each of the first plurality of time periods; combining in whole or in part, for each of the first plurality of time periods, the first heart rate variability value and the second heart rate variability value to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

2. A method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, a first heart rate variability value for each of the first plurality of time periods; determining, from the heart rate measurements taken of the user, a first respiratory sinus arrhythmia value for each of the first plurality of time periods; combining in whole or in part, for each of the first plurality of time periods, the first heart rate variability value and the first respiratory sinus arrhythmia value to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

3. A method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a sensor, taking first heart rate measurements of the user for each of the first plurality of time periods; determining, from the heart rate measurements, at least one heart rate variability value for each of the first plurality of time periods; combining in whole or in part, for each of the first plurality of time periods, the at least one heart rate variability values to create a first vagal tone value for each of the first plurality of time periods; determining the pre-determined respiration frequencies provided using first vagal tone values; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

4. A method comprising: providing the user with a first indication to breathe at a pre-determined respiration frequency for each of a first plurality of time periods, each of the first plurality of time periods having a pre-determined respiration frequency that is unique among the pre-determined respiration frequencies for the first plurality; using a heart rate sensor, taking first heart rate measurements of the user for each of the first plurality of time periods; using a respiration sensor or the guided respiration signal, taking first respiration measurements of the user for each of the first plurality of time periods; determining, from the heart rate measurements and the respiration measurements, a first phase angle value for each of the first plurality of time periods; combining in whole or in part, for each of the first plurality of time periods, the first heart rate variability values to create a first vagal tone value for each of the first plurality of time periods; comparing the created plurality of first vagal tone values to each other to determine a first optimal vagal tone value; and setting a first target respiration frequency based on a first respiration frequency from a time period corresponding to the first optimal vagal tone value, the first respiration frequency selected from the first pre-determined respiration frequency or a first actual respiration frequency.

5. A method comprising: using a heart rate sensor, taking first heart rate measurements of the user; using a respiration sensor or the guided respiration signal, taking first respiration measurements of the user; determining, from the heart rate measurements and the respiration measurements, a first phase angle value for each of the first plurality of time periods; providing the user with an indication to breathe at respiration frequencies, adjusting the indicated respiration frequency based on the sign of the first phase angle value; and stopping the adjusting of the indicated respiration frequency when sufficiently confident that the indicated respiration frequency is the best respiration frequency for the user.

6. The method of claim 5, wherein the confidence in the indicated respiration frequency being best is based on 0 degree phase angle.

7. A method comprising: initiating a feedback session by user action or software prompt; providing the user with an indication to breathe at a calculated respiratory frequency, the rate being between 4 and 8 breaths per minute; determining a biometric value from at least one of: a heart rate sensor and a respiration sensor, the biometric value(s) being heart rate variability, phase angle or respiration accuracy; creating a vagal tone value based on a difference between the biometric value and a pre-determined biometric value; providing, to the user, a first indication of vagal tone when the vagal tone value is within a first threshold amount, the first indication of vagal tone provided using a first signal from one of: a visual interface, an audio interface, and a haptic interface; measuring and recording time within a first threshold amount for each session; and providing, to the user, a second indication of vagal tone when a time-requirement for a first threshold amount is met, the second indication of vagal tone provided using a second signal from one of: a visual interface, an audio interface, and a haptic interface.

8. The method of claim 7, wherein the pre-determined value is the best recorded value from past sessions or 0 degree phase angle.

9. A method comprising: using a heart rate sensor, analyzing heart rate variability during slow breathing; comparing the delta of HRV measurements across sessions; estimating the distance of current HRV from HRV at lowest attainable delta; and providing an indication of the distance, the indication being in the form of a visual display, haptic motor or auditory.

10. The method of claim 9 further comprising weighting the average calculated in claim 12 with this measurement for one indication based on at least two metrics.

11. The method of claim 9 further comprising weighting stress level with this measurement for one indication based on at least two metrics.

12. A method comprising: using a heart rate sensor, analyzing at least one of heart rate variability and phase angle; measuring the time the at least one biometric is within a first threshold across sessions; averaging the time the at least one biometric is within a first threshold across sessions; and providing an indication of the average time that at least one biometric is within a first threshold across sessions, the indication being in the form of a visual display, haptic motor or auditory.

13. The method of claim 12 further comprising weighting the difference in delta calculated in claim 9 with this measurement for one indication based on at least two metrics.

14. The method of claim 12 further comprising weighting stress level with this measurement for one indication based on at least two metrics.

15. A method comprising: using at least one biometric sensor, taking at least two biometric measurements of the user; applying a differentiation filter to each type of biometric data; comparing correlations between all types of biometric data; and estimating lower and upper bounds of user stress level from the compared correlations using a linear form of variance.

16. The method of claim 15, wherein the correlations are calculated using a multidimensional covariance algorithm.

17. The method of claim 15, wherein correlations are based on analyzing first derivative changes in metrics.

18. The method of claim 15, wherein the lower and upper bounds are calculated using a solution to the German tank problem.

19. A method comprising: using a heart rate sensor, analyzing heart rate variability during slow breathing; comparing the delta of HRV measurements across sessions; estimating the distance of current HRV from HRV at lowest attainable delta; measuring changes in the estimated distance across sessions; analyzing trends in the measured changes in the estimated distance across sessions; and increasing recommended stress reduction goal when trends is negative in regard to health.

20. The method of claim 19 further comprising: decreasing recommended stress reduction goal when trend is positive in regard to health.

21. The method of claim 19 further comprising: weight the average calculated in claim 22 with this measurement to analyze trends based on at least two metrics.

22. The method of claim 19 further comprising: weight stress level in analysis of trends.

23. The method of claim 19 further comprising: stress reduction goal being analog for time in resonance, desired stress level baseline, athletic performance goal, or other relaxation means.

24. A method comprising: using a heart rate sensor, analyzing at least one of heart rate variability and phase angle during slow breathing, where the breath rates are between 4 and 8 breaths per minute; measuring the time the at least one biometric is within a first threshold across sessions; averaging the time the at least one biometric is within a first threshold across sessions; measuring changes in the average time the at least one biometric is within a first threshold across sessions; analyzing trends in changes in the average time the at least one biometric is within a first threshold across sessions; and increasing recommended stress reduction goal when trends is negative in regard to health;

25. The method of claim 24 further comprising: decreasing recommended stress reduction goal when trend is positive in regard to health.

26. The method of claim 24 further comprising: weight the difference in delta calculated in claim 19 with this measurement to analyze trends based on at least two metrics.

27. The method of claim 24 further comprising: weight stress level in analysis of trends.

28. The method of claim 24 further comprising: stress reduction goal being analog for time in resonance, desired stress level baseline, athletic performance goal, or other relaxation means.

29. A system comprising: a plurality of sensors configured to collect biometric data from the user; at least one processor having memory and instructions, the at least one processor being coupled to the plurality of sensors and configured to receive the biometric data from the plurality of sensors; a first indicator coupled to the at least one processor and activated by a control signal from the at least one processor, and the instructions, when executed by the at least one processor while the device is being worn by the user, causing the at least one processor to: receive first biometric data from the plurality of sensors throughout a first time interval; calculate at least one biometric value from all the received first biometric data, the biometric value being at least one heart rate, heart rate variability, respiration, blood pressure, skin conductance, and skin temperature; receive second biometric data from the plurality of sensors throughout a second time interval immediately following the first time interval; calculate the second biometric value from all the received second biometric data; compare the second biometric value to the first biometric value and when a result of the comparison is that the second biometric value is increased over the first biometric value by a threshold amount activate the first indicator to indicate an improvement of the at least one second biometric value from the at least one first biometric value; receive third biometric data from the plurality of sensors throughout a third time interval immediately following the second time interval; calculate the third biometric value from all the received third biometric data; compare the third biometric value to the second biometric value and when a result of the comparison is that the third biometric value is increased over the second biometric value by the threshold amount or the third biometric value stayed within a threshold amount from the second biometric value as long as the comparison from the first and second biometric value resulted in a first indication, activate the first indicator to indicate an improvement of the third biometric value from the second biometric value; consider the third biometric value to be a reference biometric value and the third time interval to be a reference time interval; after receiving the third biometric data, receive new biometric data from the plurality of sensors throughout a new time interval immediately following the reference time interval; calculate a new biometric value from all the received new biometric data; compare the new biometric value to the reference biometric value and when the result of the comparison is that the new biometric value is increased over the reference biometric value by the threshold amount or the new biometric value stayed within a threshold amount from the reference biometric value as long as the comparison from the third biometric value and second biometric values resulted in a first indication, activate the first indicator to indicate an improvement of the new biometric value from the reference HRV value; select the at least one new biometric value as the reference biometric value and the new time interval as the reference time interval; and repeat the receiving of new biometric data, the calculating of new biometric value, and the comparing of the new biometric value to the reference biometric value until it's desirable to end the repeating of indicating comparisons in biometric data.

30. A method comprising: using at least one sensor, measuring at least one biometric value, the biometric value being at least one of heart rate, heart rate variability, skin conductance, skin temperature, blood pressure and respiration; measuring the differences between the measured biometric values and predetermined biometric values; applying a weight to each measured difference; combining the weighted differences in a single metric; and providing an indication of wellbeing based on the single metric composed of weighted differences.

31. The method of claim 30, wherein the pre-determined biometric values are the best recorded measurements from historical data collection or 0 degrees phase difference.

32. The method of claim 30, wherein the weights can adjust based on biometrics reaching pre-determined thresholds.
